(19) United States
(12) Patent Application Publication
(10) Pub. No.: US 2011/0177164 A1
(43) Pub. Date: Jul. 21, 2011

(54) Pharmaceutical Compositions Comprising Amorphous Esomeprazole, Dosage Forms And Process Thereof

(76) Inventors: Gopal Rajan, Puram Palladam (IN); Pratik Kumar, Bihar (IN); Gour Mukherji, Haryana (IN); Pulak Kumar Metia, West Bengal (IN)

(21) Appl. No.: 13/121,281

(22) PCT Filed: Oct. 6, 2009
(86) PCT No.: PCT/IN09/00553

§ 371 Date: Mar. 28, 2011

(30) Foreign Application Priority Data
Oct. 6, 2008 (IN) 231/DEL/2008

(51) Int. Cl. A61K 9/52; A61K 9/00; A61K 31/4439; A61K 9/22; B05D 3/00; A61P 1/04

(52) U.S. Cl. 424/457; 424/400; 514/338; 424/468; 424/465; 427/2.21

(57) ABSTRACT

A stabilized pharmaceutical composition of benzimidazole compounds preferably amorphous form of esomeprazole and a process for preparing the same. The pharmaceutical compositions formulated into solid dosage forms preferably multiple unit tablet dosage forms and capsules and a method for preparing the same.

### PRIORITY CLAIM

This is a U.S. national stage of application No. PCT/IN2009/000553, filed on 6 Oct. 2009. Priority is claimed on the following application: Country: India, Application No.: 2315/DEL/2008, Filed: 6 Oct. 2008, the content of which is incorporated here by reference.

### FIELD OF THE INVENTION

The present invention, in general, relates to stabilized pharmaceutical compositions of benzimidazole compounds. More particularly, the present invention relates to stable pharmaceutical compositions of amorphous form of esomeprazole, suitable dosage forms comprising the same and a process for preparing the same.

### BACKGROUND OF THE INVENTION

Benzimidazole compounds, such as Omeprazole, Lansoprazole, Pantoprazole, Rabeprazole or single enantiomers thereof are strong inhibitors of proton pump and are widely used as therapeutic agents for stomach ulcer, duodenal ulcer, and gastro-esophageal reflux disorders. Benzimidazole compounds effectively inhibit gastric acid secretion.

U.S. Pat. No. 4,255,431 assigned to Aktiebolaget Hassle, discloses omeprazole and therapeutically acceptable salts thereof. The advantages of providing the salts of omeprazole and particularly the magnesium salt are disclosed in U.S. Pat. No. 4,738,974.

The single isomers of omeprazole are reported to be more useful in therapy when compared to the racemic omeprazole. U.S. Pat. No. 5,877,192 assigned to Astra Aktiebolag, discloses the use of the (−)-enantiomer of omeprazole (esomeprazole), or a pharmaceutically acceptable salt thereof, in the treatment of gastric acid related diseases as a means to decrease inter-individual variation in plasma levels compared to omeprazole.

European Patent No. 723437 assigned to Astra, discloses multiple unit tableted dosage forms comprising benzimidazole compound in the form of enteric-coated pellets, which are further overcoated with polymeric coating. The use of overcoating ensures surface integrity of the enteric coating pellets and protects pellets from mechanical stress, thus, prevents cracking of the enteric layer.

PCT Publication No WO2003/103637 assigned to Ranbaxy, teaches modified release multiple unit drug delivery system wherein each unit comprises inert core coated with first coating layer that includes one or more drugs and one or more rate controlling polymers and an outer layer comprising a material that is elastic and/or compressible waxy material such as PEG. The rate controlling membrane controls release of the drug over a period of 24 hours. The waxy outer layer protects the release control polymer layer from cracking during compression.

Nexium®, only commercially available dosage form in USA till date, is a capsule dosage form for oral administration containing delayed release pellets of magnesium salts of esomeprazole. Nexium® is formulated in dosage form with most stable crystalline trihydrate form of esomeprazole magnesium.

It is well known in art that the amorphous form of an active pharmaceutical ingredient is thermodynamically less stable than any crystalline form and inadvertent crystallization from an amorphous drug substance may occur. As a consequence of the higher mobility and ability to interact with moisture, heat or air, amorphous drug substances are also more likely to undergo solid-state reactions leading to degradation and subsequent loss of activity and purity. However amorphous forms exhibit better solubility and absorption characteristics.

U.S. Pat. No. 7,482,463, assigned to Ranbaxy, discloses a process for manufacturing amorphous form of esomeprazole.

Further, it has been reported that amorphous form of esomeprazole or its pharmaceutically acceptable salts are highly unstable in nature and when formulated into a dosage form leads to degradation of esomeprazole with unacceptable rise in relative substances, color change and consequent loss of its therapeutic value.

Thus there is an unmet need for a stable composition of amorphous esomeprazole. Furthermore, there exists a need for an economical and commercially viable solution for the same.

### SUMMARY OF THE INVENTION

It is an aspect of the present invention to provide a stabilized composition of a benzimidazole compound, preferably amorphous form of esomeprazole.

It is another aspect of the present invention to provide a suitable solid dosage form of amorphous form of esomeprazole comprising the stable pharmaceutical composition of the invention.

It is one another aspect of the present invention to provide a process for preparing a stabilized composition of the amorphous form of esomeprazole and suitable dosage forms thereof.

In accordance with an embodiment of the present invention, there is provided a stabilized pharmaceutical composition comprising an inert core unit, a drug layer coated over the inert core unit, wherein the drug layer consisting essentially consisting essentially of amorphous esomeprazole, a binder and a disintegrant, one or more intermediate layer consisting essentially of basic metal compounds and icing sugar with starch having a ratio from about 1:5 to about 5:1, one or more subcoating layer consisting essentially of waxy material and one or more enteric layer comprising at least one enteric polymer, and wherein the basic metal compounds are not in intimate contact with the drug in the pharmaceutical composition.

In accordance with another embodiment of the present invention, there is provided a solid dosage form of a stabilized pharmaceutical composition comprising an inert core unit, a drug layer coated over the inert core unit, wherein the drug layer consisting essentially of amorphous esomeprazole, a binder and a disintegrant, an intermediate layer consisting essentially of basic metal compounds and icing sugar with starch having a ratio from about 1:5 to about 5:1, a subcoating layer consisting essentially of waxy material, a second intermediate layer consisting essentially of basic metal compounds and icing sugar with starch having a ratio from about 1:5 to about 5:1, a second subcoating layer consisting essentially of waxy material, an enteric layer comprising at least one enteric polymer and a second enteric layer comprising at least one enteric polymer, and wherein the basic metal compounds are not in intimate contact with the amorphous esomeprazole.

In accordance with still another embodiment of the present invention, there is provided a multiple unit tablet dosage form comprising a stabilized pharmaceutical composition, wherein the pharmaceutical composition comprising an inert core unit, a drug layer coated over the inert core unit, wherein the drug layer consisting essentially of amorphous esomeprazole, a binder and a disintegrant, an intermediate layer consisting essentially of basic metal compounds, and icing sugar with starch having a ratio from about 1:5 to about 5:1, a subcoating layer consisting essentially of waxy material, a second intermediate layer consisting essentially of basic metal compounds and icing sugar with starch having a ratio from about 1:5 to about 5:1, a second subcoating layer consisting essentially of waxy material, an enteric layer comprising at least one enteric polymer, a second enteric layer comprising at least one enteric polymer; and tablet excipients, and wherein the basic metal compound is not in intimate contact with the amorphous esomeprazole in the pharmaceutical composition, and the pharmaceutical composition is free of any overcoating on the resultant enteric coated units.

In accordance with another embodiment of the present invention, the ratio of basic metallic compound to icing sugar with starch in the intermediate layers of the pharmaceutical composition is preferably between 1:3 to about 1:5.

In accordance with yet another embodiment of the present invention, there is provided a process for preparing the pharmaceutical composition comprising coating the inert core with aqueous dispersion or suspension of the drug coating layer and optionally other pharmaceutical excipients, applying the intermediate coating as an aqueous dispersion or suspension on the drug coated core, applying the aqueous dispersion or suspension of the waxy material of the subcoating layer on the resultant product, followed by optionally repeating the step of applying the intermediate and the subcoating layer on the resultant product and subsequently coating the resultant product with one or more enteric layers using an aqueous dispersion of at least one enteric polymer and drying the enteric coated product to obtain the pharmaceutical composition.

In accordance with still one another embodiment of the present invention, there is provided a process for preparing the solid dosage form containing the pharmaceutical composition, the process comprising coating the inert core with aqueous dispersion or suspension of the drug coating layer and optionally other pharmaceutical excipients, applying the intermediate coating as an aqueous dispersion or suspension on the drug coated core, applying an aqueous dispersion or suspension of waxy material of the subcoating layer on the resultant product, repeatedly applying a second intermediate and subcoating layer on the subcoated product followed by coating the resultant product with one or more enteric layer using an aqueous dispersion of at least one enteric polymer and drying the enteric coated product to obtain the pharmaceutical composition and further formulating the same into a suitable dosage form. The dosage form so obtained is optionally packaged in deoxidant-encapsulated or oxygen and water vapor permeation inhibitory packaging.

In accordance with yet one another embodiment of the present invention, there is provided a process for preparing the multiple unit tablet dosage form comprising the stabilized pharmaceutical composition, the process comprising coating the inert core with aqueous dispersion or suspension of the drug coating layer and optionally other pharmaceutical excipients, applying the intermediate coating as an aqueous dispersion or suspension on the drug coated core, applying the aqueous dispersion or suspension of waxy material of the subcoating layer, repeatedly applying a second intermediate and subcoating layer on the subcoated product followed by coating the resultant product with one or more enteric layer employing an aqueous dispersion of at least one enteric polymer, drying the enteric coated product followed by passing the same through suitable sized mesh, blending the dried product with one or more tablet excipients and compression to obtain tablets. Subsequently the multiple unit tablet dosage form is optionally packaged in a deoxidant-encapsulated or oxygen and water vapor permeation inhibitory packaging.

### DETAILED DESCRIPTION OF INVENTION

While this specification concludes with claims particularly pointing out and distinctly claiming that, which is regarded as the invention, it is anticipated that the invention can be more readily understood through reading the following detailed description of the invention and study of the included examples.

The present invention discloses a stabilized pharmaceutical composition of a benzimidazole compound, preferably an amorphous form of esomeprazole and a process for preparing the same.

Additionally the present invention discloses a solid dosage form comprising the pharmaceutical composition, specifically a multiple unit tablet dosage form. The present invention also discloses a process for producing the solid dosage form and the multiple unit tablet dosage form thereof.

“Amorphous esomeprazole” or drug as used herein includes all pharmaceutically or therapeutically acceptable salts, hydrates, solvates, and other forms of amorphous esomeprazole. Amorphous esomeprazole magnesium is the preferable form.

“Coating” as used herein also refers to layering and the two terms can be used interchangeably. The coating composition for drug layering, intermediate layering, subcoating and enteric coating layering is distinct from each other functionally.

“Stability” or stabilization or stabilized as used herein refers to preservation of the amorphous nature of the esomeprazole active pharmaceutical ingredient, and the prevention of its conversion into degradation variants, in the dosage forms of the invention.

“Related substances” as referred herein means any variant of active pharmaceutical ingredient (drug substance) resulting from a molecular or chemical or physical change in the drug substance brought about during manufacture and/or storage of the dosage form by the effect of light, temperature, pH, water, or by reaction with an excipient and/or the immediate container closure system, which may or may not have deleterious effect on the safety and efficacy of the drug product

“Active Pharmaceutical Ingredient” as mentioned herein refers to the molecular/chemical moiety, including its salt forms, responsible for bringing about a therapeutic response in a mammal.

As used herein “core” refers to the inert spherical or granular material used for coating of drug layer and subsequently intermediate, subcoating and enteric coating. Core is interchangeably used with beads, units and/or pellets. Core can be made up of material, which is water soluble, water swellable or water insoluble or any combination thereof.

“Dosage form” as used herein refers to suitable physical form like capsules, tablet, dry syrup, sachets and the like, which are convenient for administration of a drug to a patient in need of that drug. The term dosage form has been frequently interchangedly used with composition and/or formulation for the description of the present invention.

“Tablet excipients” as used herein refers to customary excipients employed in manufacturing of tablets either from granules or by direct compression technique.

“Acid resistance” as used herein refers to the property of tablet/capsule to prevent release or minimize release of the drug from dosage form when tested as per following dissolution conditions

Dissolution Media: 0.1N HCL medium,

### RPM: 100 rpm

Time: 2 hours,
and calculated as drug retained in the dosage form after designated exposure.

According to the present invention, the stabilized pharmaceutical composition comprises
(a) an inert core unit,
(b) a drug layer coated over the inert core unit, wherein the drug layer consisting essentially of amorphous esomeprazole, a binder and a disintegrant,
(c) one or more intermediate layer consisting essentially of basic metal compounds and icing sugar with starch, having a ratio from about 1:5 to about 5:1,
(d) one or more subcoating layer consisting essentially of waxy material, and
(e) one or more enteric layer comprising at least one enteric polymer,

wherein the basic metal compounds are not in intimate contact with the drug in the pharmaceutical composition.

According to the present invention the solid dosage form of the stabilized pharmaceutical composition comprises:
(a) an inert core unit,
(b) a drug layer coated over the inert core unit, wherein the drug layer consisting essentially of amorphous esomeprazole, a binder and a disintegrant,
(c) an intermediate layer consisting essentially of basic metal compounds, and icing sugar with starch,
(d) a subcoating layer consisting essentially of waxy material,
(e) a second intermediate layer consisting essentially of basic metal compounds and icing sugar with starch having,
(f) a second subcoating layer consisting essentially of waxy material,
(g) an enteric layer comprising at least one enteric polymer; and
(h) a second enteric layer comprising at least one enteric polymer, and

wherein the basic metal compound are not in intimate contact with the amorphous esomeprazole.

In accordance with the invention, the ratio of the basic metallic compound to icing sugar with starch in the intermediate layers varies from about 1:5 to about 5:1, preferably about 1:3 to about 1:5 and the pharmaceutical composition is formulated into solid dosage forms preferably tablets or capsules.

In accordance with the invention, there is provided a multiple unit tablet dosage form comprising a stabilized pharmaceutical composition, wherein the pharmaceutical composition comprising:
(a) an inert core unit;
(b) a drug layer coated over the inert core unit, wherein the drug layer consisting essentially of amorphous esomeprazole, a binder and a disintegrant;
(c) an intermediate layer consisting essentially of basic metal compounds, and icing sugar with starch having a ratio from about 1:5 to about 5:1;
(d) a subcoating layer consisting essentially of waxy material;
(e) a second intermediate layer consisting essentially of basic metal compounds, and icing sugar with starch having a ratio from about 1:5 to about 5:1;
(f) a second subcoating layer consisting essentially of waxy material;
(g) an enteric layer comprising at least one enteric polymer;
(h) a second enteric layer comprising at least one enteric polymer to obtain enteric coated units;
(i) tablet excipients; and

wherein the basic metal compound is not in intimate contact with the amorphous esomeprazole in the pharmaceutical composition, and wherein the pharmaceutical composition is free of any overcoating on the resultant enteric coated units.

### The Inert Core

According to the invention, the inert core can be water-soluble, water-swellable and water-insoluble. Non-pareil seeds (NPS) or sugar spheres exemplifies an inert core of water-soluble type. NPS are white, spherical particles of 62-92% sucrose and rest starch. NPS are commercially available from JRS Pharma, USA in various sieve sizes. Suitable examples of water swellable cores are microcrystalline cellulose spheres, commercially available from FMC Corporation under the trade name CELPHERE®. Exemplary water-insoluble inert cores are glass beads and coarse grade silicon beads. The inert core must be of sufficient density and strength to withstand stress and strain of coating process. Preferably, the inert core unit is made up of inert non-pareil sugar spheres or microcrystalline cellulose.

Preferably according to the invention, the size of NPS is selected from the following sieve sizes: 40-60 mesh sieve (250-425 μm), 40-50 mesh size (420-300 μm), 35-40 mesh sieve (425-500 μm), 30-35-mesh sieve (500-600 μm), 25-30 mesh sieve (600-710 μm), 20-25 mesh sieve (710-850 μm), 18-20 mesh sieve (850-1000 μm), 16-18 mesh sieve (1000-1180 μm), 14-16 mesh sieve (1000-1400 μm).

In a more preferred embodiment the inert cores have a diameter ranging from about 250 to 600 μm, preferably from 300 to 500 μm and most preferably 300 to 420 μm. Alternatively, a combination of the above-mentioned sieve sizes can be employed. Preferably, inert core constitutes from about 5% to about 40% by weight of the enteric-coated beads, preferably from about 10% to about 30%, still more preferably from about 15% to about 25% by weight of enteric-coated beads.

### The Drug Layer

The drug layer consisting essentially of the amorphous esomeprazole, binder and disintegrants. The inert core is coated with aqueous dispersion consisting essentially of the amorphous esomeprazole, binder, and disintegrants. According to the invention, the amorphous esomeprazole constitutes from about 10% to about 30% by weight of enteric-coated pellets.

According to the invention, the binders employed include, but are not limited to, starch, gelatin, acacia, sodium alginate, alginic acid, polyvinyl pyrrolidone (Povidone K-30), hydroxyethyl cellulose, hydroxypropyl cellulose, hydroxypropyl methylcellulose, polyacrylate, ethylcellulose, glucose, sucrose, sorbitol, mannitol, dextrose, icing sugar with starch, sodium carboxymethyl cellulose, low substituted hydroxypropyl cellulose, polyvinyl alcohol, polymethacrylates (Eudragit® NE 30 D and Eudragit® RS 30 D) and methyl cellulose, maltodextrins and chitosan derivatives. In accordance with the present invention, preferably the binder is hydroxypropyl cellulose (HPC). HPC, for binder use, is commercially available from Aqualon, USA under the brand name Klucel® EF and EXF. Binder constitute from about 0.5% to about 15%, preferably from about 1 to about 10% by weight of the enteric-coated beads,

Suitable examples of disintegrants include, but are not limited to, croscarmellose sodium, sodium starch glycolate, carboxymethylstarch, crosslinked polyvinyl pyrrolidone (Crospovidone XL10), and low-substituted hydroxypropyl cellulose (L-HPC), alginic acid, maltose, polacrilin potassium, pullulan, pregelatinized starch. Disintegrant constitute from about 1% to about 15%, preferably from about 2% to about 10% by weight of the enteric-coated beads. Crospovidone, commercially available from International Specialty Products, USA, is the preferred choice of disintegrant for drug layer.

The drug layer may further contain other pharmaceutically acceptable excipients such as solvents for dissolving the binders and/or the esomeprazole, anti-tacking agents and surfactants.

Suitable examples of surfactants include, but not limited to, polysorbate series such as, 80, 60, 40, 20; sorbitan monooleate, sodium lauryl sulphate, benzalkonium chloride, cetylpyridinium chloride. Surfactants promote the dispersion of the drug and low aqueous-solubility excipients in aqueous medium. Surfactant constitute preferably from about 0% to about 1% by weight of the enteric-coated beads. Preferably according to the invention, the surfactants include sodium lauryl sulphate, polysorbate 80, and polysorbate 20.

Suitable antitacking agents according to the invention include but are not limited to talc, silicon dioxide and magnesium stearate. Antitacking agents constitute preferably from about 0% to about 1% by weight of the enteric-coated beads.

The drug layer is applied to the inert core by any conventional techniques known in the art, such as, pan coating, roto-granulation or fluidized bed coating. During such coating operations the drug is dispersed or dissolved or suspended in an organic or aqueous solvent, which can also contain above-mentioned excipients. The solvent system used for processing the drug layer can be aqueous or non aqueous. Appropriate non-aqueous solvents can be alcoholic, such as, methanol, ethanol, isopropyl alcohol (IPA); hydro-alcoholic, such as, water-IPA; organic solvents, such as, acetone, methylene chloride or any combination of those mentioned above. In a preferred embodiment according to the invention, aqueous solvent, purified water is employed and drug layering is carried out in fluid bed processor fitted with wurster apparatus.

Drug layering constitutes from about 10% to about 25% by weight enteric-coated beads.

### Intermediate Layer

The composition of the invention contains one or more intermediate layers, which may alternate with one or more subcoating layers. The composition of the first and second intermediate layers according to the invention may be similar or different.

The intermediate layer is critical for the stabilized composition of the present invention as it is instrumental in securing the stability of the composition of the present invention. The composition of the intermediate layer reduces the formation of related substances in the dosage form and in concert with processing conditions preserves and conserves the amorphous nature of the esomeprazole in the dosage form.

The intermediate layer consisting essentially of a basic metallic compound and icing sugar with starch. The basic metallic compound is selected from metal carbonates, and metal oxides. Other suitable examples of the basic metallic compound include, but not limited to, magnesium carbonate, calcium carbonate, magnesium oxide, calcium oxide, magnesium hydroxide, sodium carbonate, sodium bicarbonate, sodium hydroxide, magnesium metasilicate aluminate, magnesium silicate aluminate and magnesium aluminate. According to the present invention, magnesium oxide is the preferable basic metallic compound employed in the intermediate layer.

According to the invention, in order to ensure uniform distribution of the basic metallic compound in the intermediate layer, it is essential to incorporate a carrier in the coating suspension/dispersion containing the basic metallic compound. The most preferred carrier for the intermediate layer is icing sugar with starch. The basic metallic compound, for each of the intermediate layers, constitute from about 0.5% to about 7%, preferably from about 1% to about 3% by weight of the enteric-coated units. Similarly for each intermediate layer, the carrier for the basic inorganic compound in the intermediate layer constitutes from about 1% to about 12%, preferably from about 3% to about 9% by weight of the enteric-coated beads.

According to the invention, the basic metallic compound is not mixed with the drug nor are they in uniform contact with one another. Presence of the basic metallic compound, preferably, magnesium oxide in combination with icing sugar with starch stabilizes the composition of invention, as related substances are restricted to at a very low level.

The intermediate layer may further contain antitacking agents, suspending agents, and surfactants to facilitate the processing of layering or coating operation. Antitacking agents such as talc constitute preferably from about 0.5% to about 5% by weight of the enteric-coated beads.

In accordance with the invention, the ratio of basic metallic compounds, such as, magnesium oxide to icing sugar with starch, in the intermediate layers is also vital for the stability of the composition of the invention. Preferable ratio of the basic metallic compound to icing sugar with starch varies from about 1:5 to about 5:1, preferably from about 1:3 to about 1:5.

Fluid bed processor (FBP) fitted with necessary apparatus is used to carry out application of the intermediate layer on drug-coated beads. Operating parameters of the FBP are well known to the skilled artisan.

Each intermediate layer constitutes from about 2% to about 20% by weight of enteric-coated beads.

### Subcoating Layer

The pharmaceutical composition of the present invention further contains one or more layer of subcoating, which may alternate, with one or more intermediate layers. The composition forming the first and second subcoating layers according to the invention may be similar or different.

The subcoating layer of the pharmaceutical composition of the present invention consisting essentially of waxy substances. Suitable examples of the waxy substances include, but are not limited to, compounds of PEG series such as polyethylene glycol 4000, 6000; glyceryl behenate, stearic acid and Gelucire® or combinations thereof. Preferably the waxy material for the subcoating layer is polyethylene glycol 6000.

The subcoating layer is applied to the intermediate layered units by coating in suitable equipment like coating pan, coating granulator and fluid bed processor using water or organic solvents like isopropyl alcohol. Optionally, other additives, such as, plasticizer, anti-tacking and anti-static agents may be used along with waxy substances. Preferably, the waxy substance constitutes 90% to 100% of waxy layer and 0.5% to 10% by weight of the enteric-coated beads/units.

Each subcoating layer constitutes from about 1% to about 15% by weight of the enteric-coated beads.

### Enteric Coating Layer

According to the invention the pharmaceutical composition contains one or more enteric layers, of same or different enteric polymers, disposed on the subcoated beads. The enteric layer consisting essentially of enteric polymers. Besides, enteric layer may also contain plasticizers, opacifying agents, antitacking agents, stabilizers, antifoaming agents, surfactants, antistatic agent, colorants, and pigments to enhance the processability, functionality and elegance of the said layers.

The polymers useful in the enteric layer include, but not limited to, cellulose acetate phthalate, cellulose acetate trimellitate, hydroxypropylmethylcellulose acetate succinate (HPMCAS), hydroxypropyl methylcellulose phthalate (HPMCP), shellac, polyvinylacetate phthalate, acrylic acid polymers & copolymers, and methacrylic acid polymers & co-polymers or any combinations thereof. Preferably according to the present invention, the enteric polymers are methacrylic acid copolymers or HPMCP or combination thereof. Methacrylic acid copolymer is commercially available under the brand name Eudragit® L30 D55.

The enteric polymers constitute from about 10% to about 50% by weight of enteric-coated beads, preferably, from about 20% to about 30%. The plasticizers, which impart flexibility to the enteric layer constitute from about 1% to about 15%, preferably from about 3% to about 9% by weight of the enteric-coated beads, all percentages based on weight-by-weight basis. Exemplary, plasticizers are triacetin, citric acid esters, phthalic acid esters, dibutyl sebacate, polyethylene glycol and cetyl alcohol.

Various antitacking and antistatic agents according to the invention include, but are not limited to, talc, silicon dioxide, metallic salts of stearic acid and glyceryl monostearate. Optimum concentration of these agents in the enteric layer is well known to the skilled artisan.

Optionally, the enteric layer may also include 0.1% to about 2% of surfactant. The enteric layers are deposited on the subcoated beads in suitable equipment like a fluid bed processor using aqueous or non-aqueous solvents. The solvent system employed for processing of enteric layer and is preferably aqueous, but it can be a combination of aqueous and organic solvent. The enteric layer is essential to prevent degradation of the acid-susceptible esomeprazole drug during its transit from the acidic environment of stomach. Preferably according to the invention, each enteric-coated layer constitutes from about 15% to about 40% by weight of enteric-coated beads.

According to the invention, the waxy and enteric coating can be carried out using fluid bed processor.

Enteric-coated units are either filled into suitable size capsules or formulated into suitable size tablets. For formulation of enteric-coated units into tablets, said units are combined with necessary tableting excipients, which afford essential attributes, to enteric-coated beads/pellets, for compression into tablets.

### Tablet Formation

For the formulation of tablets various tableting excipients are employed which include but are not limited to, diluents, binders, cushioning agents, surfactants, disintegrants, lubricants, sweeteners, anti-tacking agents, flavoring agents antiadherants and glidants.

The tablet excipients are added before compressing into tablets. Diluents employed are preferably directly compressible diluents, which impart necessary flow and compressibility property to the tablet mix to facilitate the process of tablet manufacturing.

The enteric-coated beads are transformed into tablets after combining with necessary tableting excipients. The enteric-coated beads constitute from about 25% to about 60% by weight of tablet weight. Ratio of enteric-coated beads to tableting excipients preferably varies from 2:3 to 3:2.

Various tableting excipients are directly compressible diluent or simple diluents, binders, disintegrants, cushioning agents, surfactants, anti-adherents, glidants and lubricants. The directly compressible diluents employed include microcrystalline cellulose (Avicel PH 102 from Signet), Cellactose (a combination of MCC and lactose from Meggle, Germany), Pharmatose DCL-40 (combination of anhydrous lactose and lactitol from DMV, Netherlands), Di-pac (sucrose and dextrin from American sugar, USA), dicalcium phosphate (Emcompress® from Edward mendell, USA). Preferably the diluent for the present invention is Avicel® PH102. Avicel® PH102 is favorable because it possesses binder property also. The diluent constitutes from about 30 to about 60% by weight of tablet weight.

Suitable examples of binders in the present invention include methylcellulose, hydroxypropyl cellulose, polyvinyl pyrrolidone, microcrystalline cellulose and hydroxypropyl methylcellulose and low substituted hydroxypropyl cellulose. Binders constitute from about 1% to about 10%, by weight of tablet weight.

Useful examples of disintegrants including superdisintegrants include, but are not limited to, croscarmellose sodium, sodium starch glycolate, crosslinked polyvinyl pyrrolidone (crospovidone), and low-substituted hydroxypropyl cellulose (L-HPC), alginic acid, maltose, polacrilin potassium, pullulan, pregelatinized starch. Preferred disintegrant is crosslinked polyvinyl pyrrolidone (crospovidone). Disintegrant constitute from about 1% to about 20%, preferably from about 5% to about 15% by weight of tablet weight.

In order to enhance the plasticity of enteric-coated pellets, a cushioning agent, preferably polyethylene glycol is employed in concentration of about 5% to about 30% by weight of tablet weight. Suitable examples of lubricants include calcium and magnesium salts of stearic acid, glyceryl behenate and sodium stearyl fumarate. Preferred lubricant is sodium stearyl fumarate. The lubricant is used in an amount of about 0.1% to about 5% by weight of tablet weight. The tablet composition may also contain suitable glidants and antiadherants, as known to skilled artisan, in customary concentration. The tableting excipients are blended homogeneously with enteric-coated pellets in a suitable blender, like twinshell blender, before being compressed in rotary tableting machine equipped with appropriate punches.

The pharmaceutical composition of the invention in tablet form may optionally be film coated to improve its elegance. Film coating materials usually include water-soluble polymers like hydroxypropyl methylcellulose, polyethylene glycol; coloring agents, and pigments. Opadry® pink is the commercially available film coating material from Colorcon, USA. Opadry® pink constitute of polyethylene glycol, hypromellose, iron oxide red, and titanium dioxide.

A solvent system is used for processing the drug layer or other coatings/layers wherein the system can be aqueous or non aqueous. Appropriate non-aqueous solvents can be alcoholic, such as, methanol, ethanol, isopropyl alcohol (IPA): hydro-alcoholic, such as, water-IPA; organic solvents, such as, acetone, methylene chloride or any combination of those mentioned above.

According to the present invention, the process for preparing the pharmaceutical composition comprises

(a) coating the inert core with aqueous an dispersion or suspension of the drug coating layer and optionally other pharmaceutical excipients,

(b) applying the intermediate coating as an aqueous dispersion or suspension on the drug coated core,

(c) applying an aqueous dispersion or suspension of the waxy material of the subcoating layer on the resultant product;

(d) optionally repeating step (b) and (c) on the subcoated product obtained in step (c);

(e) coating the resultant product with one or more enteric layer using an aqueous dispersion of at least one enteric polymer; and

(f) drying the enteric coated product to obtain the pharmaceutical composition.

Alternatively, according to the present invention, the process for preparing the pharmaceutical composition in a suitable dosage form comprises:
(a) coating the inert core with an aqueous dispersion or suspension of the drug coating layer and optionally other pharmaceutical excipients;
(b) applying the intermediate coating as an aqueous dispersion or suspension on the drug coated core;
(c) applying an aqueous dispersion or suspension of the waxy material of the subcoating layer on the resultant product;
(d) repeating step (b) and (c) on the subcoated product obtained in step (c);
(e) coating the resultant product with one or more enteric layer using an aqueous dispersion of at least one enteric polymer; and
(f) drying the enteric coated product to obtain the pharmaceutical composition and optionally packaging the same in deoxidant-encapsulated or oxygen and water vapor permeation inhibitory packaging.

Furthermore, according to the present invention, the process for preparing the multiple unit tablet dosage form containing the stabilized pharmaceutical composition comprises:
(a) coating the inert core with an aqueous dispersion or suspension of the drug coating layer and optionally other pharmaceutical excipients;
(b) applying the intermediate coating as an aqueous dispersion or suspension on the drug coated core;
(c) applying an aqueous dispersion or suspension of the waxy material of the subcoating layer on the resultant product of step b;
(d) repeating the step (b) and (c) on the subcoated product obtained in step (c),
(e) coating the resultant product with the one or more enteric layer employing an aqueous dispersion of at least one enteric polymer;
(f) drying the enteric coated product followed by passing the same through suitable sized mesh;
(g) blending the dried product of step (f) with one or more tablet excipients; and
(h) compressing the blend obtained in step (g) to obtain tablets and optionally packaging the same in deoxidant-encapsulated or oxygen and water vapor permeation inhibitory packaging.

According to an alternate embodiment of the invention the dried enteric coated units are passed through a suitable size mesh and the enteric coated units are filled in suitable size capsules. Alternatively, the enteric coated beads of step (g) are blended with tablet excipients including diluents, disintegrant, binders, surfactants, cushioning agent, lubricant, anti-adherants and glidants, followed by compressing the blend on a tableting machine using suitable tooling to obtain tablets of suitable size.

In order to improve the stability of amorphous esomeprazole in the dosage form, the dosage form is packed in the package, which prevents the permeation of oxygen and water vapor, package wherein reactive oxygen is replaced with non-reactive gases, and package encapsulated with deoxidizer. The tablets or capsules thus obtained are packaged in deoxidant-encapsulated or oxygen and water vapor permeation inhibitory packaging.

The stable pharmaceutical composition comprising amorphous esomeprazole and said composition, either in capsule or compressed tablet form, is resistant to dissolution in acidic dissolution media. However, the dissolution profile of the composition of the invention is in absolute conformity with the established standards of USP and EP.

Despite exposure to considerable compression pressure during the tableting operation, the tablets of present invention exhibit outstanding acid resistance property.

The composition of the invention is useful and recommended in the treatment of gastro-esophageal reflux disease, duodenal ulcer and Zollinger-Ellison syndrome. The dose-requirement of the drug is worked out on the basis of the severity of the disease and other factors. A typical dose varies from 20 mg to 40 mg once daily for 4 to 8 weeks only.

The following non-limiting examples illustrate specific embodiments of the present invention. They are, however, not intended to be limiting the scope of the present invention in any way.

### Example 1

### Delayed Release Amorphous Esomeprazole Composition/Formulation (Tablets)

TABLE 1

| | Examples | | | | | |
|---|---|---|---|---|---|---|
| Subject | 1T | 2T | 3T | 4T | 5T | Ref. 1 |
| Inert core | | | | | | |
| Non pareil seeds (40/50) | 40 | 40 | 40 | 40 | 40 | 40 |
| Total inert core | 40 | 40 | 40 | 40 | 40 | 40 |
| Drug Layer | | | | | | |
| Amorphous Esomeprazole | 45.02 | 45.02 | 45.02 | 45.02 | 44.53 | 45.02 |
| Hydroxypropyl cellulose | 15.0 | 10.0 | 15.0 | 15.00 | 15.00 | 3.20 |
| Povidone K30 | — | — | — | — | — | 20.0 |
| Crospovidone | — | — | — | 12.50 | 12.50 | — |
| Purified water* | qs | qs | qs | qs | qs | qs |
| Total Drug Layer | 60.02 | 55.02 | 60.02 | 72.52 | 72.03 | 68.22 |

*Evaporates during processing

TABLE 2

| | 1T | 2T | 3T | 4T | 5T | Ref. 1 |
|---|---|---|---|---|---|---|
| Intermediate Layer-I | | | | | | |
| Magnesium oxide | 10.17 | 9.65 | 26.99 | 11.36 | 5.02 | — |
| Icing sugar with starch | 30.52 | 28.97 | 80.72 | 34.15 | 15.05 | — |
| Hydroxyethyl cellulose | — | — | — | — | — | 12.40 |
| Talc | 4.75 | 4.52 | 12.55 | 5.31 | 2.33 | 18.54 |
| Purified water* | qs | Qs | qs | qs | qs | qs |
| Total Intermediate | 45.44 | 43.14 | 120.26 | 50.82 | 22.41 | 30.94 |
| Layer- | | | | | | |
| Subcoating Layer-I | | | | | | |
| PEG 6000 | 13.58 | 12.88 | 25.26 | 15.25 | 6.724 | — |
| Purified water* | qs | Qs | qs | qs | qs | — |
| Total Subcoating | 13.58 | 12.88 | 25.26 | 15.25 | 6.724 | — |
| Layer I | | | | | | |
| Intermediate Layer II | | | | | | |
| Magnesium oxide | — | — | — | — | 6.324 | — |
| Icing sugar with starch | — | — | — | — | 18.974 | — |
| Talc | — | — | — | — | 2.93 | — |
| Purified water* | — | — | — | — | qs | — |
| Total Intermediate | — | — | — | — | 28.234 | — |
| Layer- | | | | | | |
| Subcoating Layer-II | | | | | | |
| PEG 6000 | — | — | — | — | 8.470 | — |
| Purified water* | — | — | — | — | qs | — |
| Total Subcoating | — | — | — | — | 8.470 | — |
| Layer II | | | | | | |

*Evaporates during processing

TABLE 3

| | 1T | 2T | 3T | 4T | 5T | Ref. 1 |
|---|---|---|---|---|---|---|
| Enteric coating I | | | | | | |
| Eudragit L30 D55 | 67.50 | 67.50 | 67.50 | 67.50 | 109.35 | 6.71 |
| Glyceryl Monostearate | 3.38 | 3.38 | 3.38 | 3.38 | 5.468 | — |
| Polysorbate 80 | 1.35 | 1.35 | 1.35 | 1.35 | 2.186 | — |
| PEG 400 | 10.13 | 10.13 | 10.13 | 10.13 | 16.402 | — |
| TEC | — | — | — | — | — | 2.01 |
| Talc | — | — | — | — | — | 2.01 |
| Purified water* | qs | Qs | qs | qs | qs | qs |
| Total enteric coating I | 82.36 | 82.36 | 82.36 | 82.36 | 133.40 | 10.73 |
| Enteric coating II | | | | | | |
| HPMCP 55 | 67.0 | 53.0 | 60.0 | 64.0 | 84.89 | 47.37 |
| PEG 400 | 6.7 | 5.3 | 6.0 | 4.8 | 8.49 | — |
| TEC | — | — | — | — | — | 14.21 |
| Talc | — | — | — | — | — | 14.21 |
| Crospovidone XL10 | — | — | — | 1.60 | — | — |
| Acetone* | qs | Qs | qs | qs | qs | qs |
| Purified water* | qs | Qs | qs | qs | qs | qs |
| Total enteric coating II | 73.7 | 58.3 | 66.0 | 70.40 | 93.38 | 75.79 |

*Evaporates during processing

TABLE 4

| | Examples | | | | | |
|---|---|---|---|---|---|---|
| Subject | 1T | 2T | 3T | 4T | 5T | Ref. 1 |
| Tablet | | | | | | |
| formulation | | | | | | |
| Enteric coated | 340.00 | 310.00 | 400.00 | 365.00 | 404.64 | 228.27 |
| II Beads | | | | | | |
| Microcrystal- | 360.95 | 440.95 | 400.95 | 436.0 | 446.28 | 460.18 |
| line cellulose | | | | | | |
| (Avicel PH102) | | | | | | |
| Crospovidone | 10.50 | 10.50 | 10.50 | 10.50 | 10.50 | 10.50 |
| XL | | | | | | |
| PEG 6000 | 87.50 | 87.50 | 87.50 | 87.50 | 87.50 | — |
| Sodium Stearyl | 1.05 | 1.05 | 1.05 | 1.05 | 1.05 | 1.05 |
| Fumarate | | | | | | |
| Film Coating | | | | | | |
| Opadry pink | 20.8 | 22.25 | 23.5 | 27 | 28.5 | 35 |
| Cutina HR PH | 3.12 | 3.25 | 3.5 | — | — | — |
| Purified water* | qs | Qs | qs | qs | qs | qs |
| Total Tablet | 823.92 | 875.5 | 927.0 | 927.05 | 978.47 | 735.0 |
| weight | | | | | | |

*Evaporates during processing

### Process for the Preparation of Delayed Release Amorphous Esomeprazole Composition/Formulation (for Example 1T to 4T)

### Manufacturing Procedure:

The entire manufacturing process was divided into three major steps:
1. Manufacturing of enteric-coated pellets (units) of esomeprazole.
2. Manufacturing of MUPS-multi-unit pellet systems of enteric-coated pellets of the esomeprazole (i.e. compression of enteric-coated pellets into tablets using suitable diluents).
3. Film coating of compressed tablets.

### Stage I

### Drug Layering

(a) Hydroxypropyl cellulose and amorphous esomepraozle magnesium was dispersed in sufficient quantity of water.
(b) Sugar spheres (NPS) were loaded in fluid bed processor and coated with dispersion of stage I (a).
(c) Drug layered pellets (inert core) were dried and passed through suitable size mesh.

### Stage II

### Intermediate Coating

a) Icing sugar with starch, magnesium oxide and talc were suspended in sufficient quantity of purified water, and passed through suitable size mesh.
b) Drug coated pellets of I (c) were coated with suspension of II (a) in fluid bed processor.
c) Intermediate-coated pellets were dried and passed through suitable size mesh.

### Stage III

### Sub-Coating

a) PEG 6000 was dispersed in sufficient quantity of water under stirring,
b) Pellets of II (c) were coated with dispersion of III (a) in fluid bed processor, and subsequently dried and passed through suitable size mesh.

### Stage IV

### Enteric Coating-I

a) Emulsion of glycerol monostearate was prepared in warm purified water with the help of polysorbate 80.
b) Polyethylene glycol was dispersed in suitable quantity of purified water and added to emulsion of IV (a).
c) Dispersion of IV (b) was added to dispersion of Eudragit L30 D55 under constant stirring.
d) Subcoated pellets of III (b) were loaded into fluid bed processor and coated with dispersion of IV (c).
e) Enteric-coated pellets were dried and passed through suitable size mesh.

### Stage V

### Enteric Coating-II

a) Polyethylene glycol 400 was dispersed in the mixture of acetone and water.
b) Hydroxypropyl methylcellulose phthalate 55 was added to dispersion of V (a).
c) Enteric-coated pellets of IV (e) were loaded in fluid bed processor and coated with dispersion of V (b), subsequently, pellets were dried and passed through suitable size mesh.

### Stage VI

### Tablet Formation

a) Microcrystalline cellulose, sodium stearyl fumarate, polyethylene glycol 6000 and crospovidone XL were passed through suitable size mesh and mixed with enteric-coated pellets of V(c) in a conta blender for 15 minutes.
b) Blend of VI (a) was compressed into tablets of suitable dimensions using tableting machine.

### Stage VII

### Film Coating

a) Opadry pink was dispersed in purified water under stiffing to get a homogenous dispersion.
b) Compressed tablets of VI (b) were film coated in coating pan.

### Process for Preparation of Example 5T

Process for preparation of example 5T is essentially the same as mentioned under process for examples 1T to 4T, except where applicable extra intermediate and sub-coating were applied as mentioned in the composition of 5T above.

### Process for Preparation of Reference Example 1

### Stage I

### Drug Layering

a) Hydroxypropyl cellulose, Povidone K30 and the amorphous esomepraozle magnesium were dispersed in sufficient quantity of water and isopropyl alcohol.
b) Sugar spheres (NPS) (inert core) were loaded in fluid bed processor and coated with dispersion of stage I (a).
c) Drug layered pellets were dried and passed through suitable size mesh.

### Stage II

### Intermediate Coating

a) Hydroxyethyl cellulose and talc were suspended in sufficient quantity of purified water, and passed through suitable size mesh.
b) Drug coated pellets of I (c) were coated with suspension of II (a) in fluid bed processor.
c) Intermediate-coated pellets were dried and passed through suitable size mesh.

### Stage III

### Enteric Coating-I

a) Triethyl citrate, and talc were dispersed in suitable quantity of purified water.
b) Dispersion of III (a) was added to dispersion of Eudragit L30 D55 under constant stirring.
c) Subcoated pellets of II (c) were loaded into fluid bed processor and coated with dispersion of III (b).
d) Enteric-coated pellets were dried and passed through suitable size mesh.

### Stage IV

### Enteric Coating-II

a) Triethyl citrate and talc was dispersed in the mixture of acetone and water.
b) Hydroxypropyl methylcellulose phthalate 55 was added to dispersion of IV (a).
c) Enteric-coated pellets of III (d) were loaded in fluid bed processor and coated with dispersion of IV (b), subsequently pellets were dried and passed through suitable size mesh.

### Stage V

### Tablet Formation

a) Microcrystalline cellulose, sodium stearyl fumarate and crospovidone XL were passed through suitable size mesh and mixed with enteric-coated pellets of IV(c) in a conta blender for 15 minutes.
b) Blend of V (a) was compressed into tablets of suitable dimensions using tableting machine.

### Stage VI

### Film Coating

Compressed tablets of V (b) were film coated, using above-mentioned coating composition, in coating pan.

### Example 6

### Stability Testing

Compositions of the invention and reference example 1, were subjected to accelerated stability testing at 40° C.±2° C., 75%±5% RH in closed containers (HDPE Bottles) for 1 to 3 months, and in open petri dishes for 7 to 14 days. The results were analyzed for the total Related Substances. Results are set out in table 5 below:

TABLE 5
(a) Stability Testing in closed container (HDPE Bottles)

| | Composition of invention | | | | Composition of reference | | | |
|---|---|---|---|---|---|---|---|---|
| | (EX. 5) | | | | Ex. 1 | | | |
| | Initial | 1 Mo | 2 Mo | 3 Mo | Initial | 1 Mo | 2 Mo | 3 Mo |
| Total RS | 0.17 | 0.36 | 0.47 | 0.91 | 0.19 | 1.62 | 2.76 | — |

(b) Stability Testing in Open petridishes

| | Composition of invention | | | Composition of reference | | |
|---|---|---|---|---|---|---|
| | (EX. 5) | | | Ex. 1 | | |
| | Initial | 7 Days | 14 Days | Initial | 7 Days | 14 Days |
| Total RS | 0.18 | 0.38 | 0.77 | 0.19 | 6.683 | — |

As clearly evident from the test results (table 5), the composition of invention has unexpectedly reduced the formation of total related substances as compared with the composition of reference. Experimental data profoundly testify to the fact that magnesium oxide in intermediate layer along with icing sugar with starch is critical for stabilizing the composition of the invention. Furthermore, the composition of the invention retains the amorphous nature of the esomeprazole after exposure to stringent stability testing conditions.

### Example 7

### Acid Resistant Test

Acid resistance test for the multiple unit tablets and enteric coated tablets of the composition of the present invention was carried out and the results were compared with data of omeprazole magnesium multiple unit tablet as reported in Ex. 3 of EP 1078628 assigned to Astra Zeneca UK.

| | Acid Resistance | Acid Resistance |
|---|---|---|
| Example | (%) Pellets | (%) Tablets |
| 5T | 98.03 | 99.09 |
| 4T | ND | 100.00 |
| Ex. 3 of EP | 98.0 | 82.0 |
| 1078628 | | |

As clearly evident from the above-mentioned data, acid resistance for the tablet of the present invention is outstanding & is more than 20% higher than the Ex 3 (with overcoating) of EP1078628.

Many modifications and variations of this invention can be made without departing from the scope of the present invention as will be apparent to those skilled in the art. Invention is to be limited only by the terms of the appended claims, along with full scope of the equivalents to which claims are entitled.

We claim:

1. A stabilized pharmaceutical composition comprising:
(a) an inert core unit;
(b) a drug layer coated over the inert core unit, wherein the drug layer consisting essentially of amorphous esomeprazole, a binder and a disintegrant;
(c) one or more intermediate layer consisting essentially of a basic metal compound and icing sugar with starch, having a ratio from about 1:5 to about 5:1;
(d) one or more subcoating layer consisting essentially of a waxy material;
(e) one or more enteric layer comprising at least one enteric polymer; and
wherein the basic metal compound is not in intimate contact with the drug in the pharmaceutical composition.

2. The stabilized pharmaceutical composition according to claim 1, wherein the pharmaceutical composition is in a solid dosage form.

3. The stabilized pharmaceutical composition according to claim 1, wherein the pharmaceutical composition is formulated into tablets or capsules.

4. A solid dosage form of a stabilized pharmaceutical composition comprising:
(a) an inert core unit;
(b) a drug layer coated over the inert core unit, wherein the drug layer consisting essentially of amorphous esomeprazole, a binder and a disintegrant;
(c) an intermediate layer consisting essentially of a basic metal compound, and icing sugar with starch having a ratio from about 1:5 to about 5:1;
(d) a subcoating layer consisting essentially of a waxy material;
(e) a second intermediate layer consisting essentially of a basic metal compound and icing sugar with starch having a ratio from about 1:5 to about 5:1;
(f) a second subcoating layer consisting essentially of a waxy material;
(g) an enteric layer comprising at least one enteric polymer;
(h) a second enteric layer comprising at least one enteric polymer; and
wherein the basic metal compound is not in intimate contact with the amorphous esomeprazole.

5. A multiple unit tablet dosage form comprising a stabilized pharmaceutical composition, wherein the pharmaceutical composition comprising:
(a) an inert core unit;
(b) a drug layer coated over the inert core unit, wherein the drug layer consisting essentially of amorphous esomeprazole, a binder and a disintegrant;
(c) an intermediate layer consisting essentially of a basic metal compound, and icing sugar with starch having a ratio from about 1:5 to about 5:1;
(d) a subcoating layer consisting essentially of a waxy material;
(e) a second intermediate layer consisting essentially of a basic metal compound, and icing sugar with starch having a ratio from about 1:5 to about 5:1;
(f) a second subcoating layer consisting essentially of a waxy material;
(g) an enteric layer comprising at least one enteric polymer;
(h) a second enteric layer comprising at least one enteric polymer;
(i) tablet excipients; and
wherein the basic metal compound is not in intimate contact with the amorphous esomeprazole in the pharmaceutical composition, and wherein the pharmaceutical composition is free of any overcoating on the resultant enteric coated units.

6. The pharmaceutical composition according to claim 1, wherein the basic metal compound comprises from magnesium carbonate, calcium carbonate, magnesium oxide, magnesium hydroxide, sodium carbonate, sodium bicarbonate, sodium hydroxide or a combination thereof.

7. The pharmaceutical composition according to claim 6, wherein the basic metal compound is magnesium oxide.

8. The pharmaceutical composition according to claim 1, wherein the ratio of basic metallic compound to icing sugar with starch in one or more intermediate layer varies from about 1:3 to about 1:5.

9. A process for preparing the pharmaceutical composition according to claim 1, said process comprising:
(a) coating the inert core with the aqueous dispersion or suspension of the drug coating layer and optionally other pharmaceutical excipients;
(b) applying the intermediate coating as an aqueous dispersion or suspension on the drug coated core;
(c) applying the aqueous dispersion or suspension of the waxy material of the subcoating layer on the resultant product;
(d) optionally repeating step (b) and (c) on the subcoated product obtained in step (c);
(e) coating the resultant product with one or more enteric layer using an aqueous dispersion of at least one enteric polymer; and
(f) drying the enteric coated product to obtain the pharmaceutical composition.

10. A process for preparing the solid dosage form of a stabilized pharmaceutical composition according to claim 4, said process comprising:
(a) coating the inert core with the aqueous dispersion or suspension of the drug coating layer and optionally other pharmaceutical excipients;
(b) applying the intermediate coating as an aqueous dispersion or suspension on the drug coated core;
(c) applying the aqueous dispersion or suspension of the waxy material of the subcoating layer on the resultant product;
(d) repeating step (b) and (c) on the subcoated product obtained in step (c);
(e) coating the resultant product with one or more enteric layer using an aqueous dispersion of at least one enteric polymer; and
(f) drying the enteric coated product to obtain the pharmaceutical composition and
(g) optionally, packaging the tablets in deoxidant-encapsulated or oxygen and water vapor permeation inhibitory packaging.

11. A process for preparing the multiple unit tablet dosage form comprising the stabilized pharmaceutical composition according to claim 5, said process for preparing the multiple unit tablet dosage form comprising:
(a) coating the inert core with the aqueous dispersion or suspension of the drug coating layer and optionally other pharmaceutical excipients;
(b) applying the intermediate coating as an aqueous dispersion or suspension on the drug coated core;
(c) applying the aqueous dispersion or suspension of the waxy material of the subcoating layer on the resultant product of step (b);
(d) repeating the step (b) and (c) on the subcoated product obtained in step (c);
(e) coating the resultant product with one or more enteric layer employing an aqueous dispersion of at least one enteric polymer;
(f) drying the enteric coated product followed by passing the same through suitable sized mesh;
(g) blending the dried product of step (f) with one or more tablet excipients; and
(h) compressing the blend obtained in step (g) to obtain tablets; and
(i) optionally, packaging the tablets in deoxidant-encapsulated or oxygen and water vapor permeation inhibitory packaging.

12. The process for preparing multiple unit dosage form according to claim 11, wherein the one or more tablet excipient is selected from diluents, disintegrants, binders, surfactants, cushioning agents, lubricants and glidants.

13. The pharmaceutical composition according to claim 4, wherein the basic metal compound comprises from magnesium carbonate, calcium carbonate, magnesium oxide, magnesium hydroxide, sodium carbonate, sodium bicarbonate, sodium hydroxide or a combination thereof.

14. The pharmaceutical composition according to claim 13, wherein the basic metal compound is magnesium oxide.

15. The pharmaceutical composition according to claim 4, wherein the ratio of basic metallic compound to icing sugar with starch in the one or more intermediate layer varies from about 1:3 to about 1:5.

16. The pharmaceutical composition according to claim 5, wherein the basic metal compound comprises from magnesium carbonate, calcium carbonate, magnesium oxide, magnesium hydroxide, sodium carbonate, sodium bicarbonate, sodium hydroxide or a combination thereof.

17. The pharmaceutical composition according to claim 16, wherein the basic metal compound is magnesium oxide.

18. The pharmaceutical composition according to claim 5, wherein the ratio of basic metallic compound to icing sugar with starch in the one or more intermediate layer varies from about 1:3 to about 1:5.
